(19) United States
(12) Patent Application Publication
(10) Pub. No.: US 2022/0313422 A1
(43) Pub. Date: Oct. 6, 2022

(54) INTRAOCULAR LENS SYSTEM, INTRAOCULAR LENS AND CILIAR BODY IMPLANT

(71) Applicant: Carl Zeiss Meditec AG, Jena (DE)

(72) Inventors: Jan Buchheister, Jena (DE); Peter Klopfleisch, Jena (DE); Lothar Müller, Ottendorf (DE); Ralph Semmler, Jena (DE); Enrico Geißler, Jena (DE)

(21) Appl. No.: 17/842,124

(22) Filed: Jun. 16, 2022

Related U.S. Application Data
(63) Continuation of application No. PCT/EP2020/087157, filed on Dec. 18, 2020.

(30) Foreign Application Priority Data
Dec. 20, 2019 (DE) 10 2019 135 508.7

(51) Int. Cl. A61F 2/16; A61F 2/48

(52) U.S. Cl. CPC A61F 2/1624; A61F 2/482; A61F 2002/1682; A61F 2210/009; A61F 2250/0096

(57) ABSTRACT

An intraocular lens system for implantation in an eye is provided. The intraocular lens system includes a ciliary body implant having a passive ciliary signal element, the ciliary body implant being implantable in the eye such that the ciliary signal element provides a ciliary signal in response to a movement of the ciliary muscle of the eye. The intraocular lens system also includes an intraocular lens having a sensor element for receiving the ciliary signal. The ciliary body implant and the intraocular lens are formed separately from each other and the intraocular system is configured to control a refractive effect of the intraocular lens that is dependent on the ciliary signal received from the sensor element.

### CROSS REFERENCE TO RELATED APPLICATIONS

This application is a continuation application of international patent application PCT/EP2020/087157, filed Dec. 18, 2020, designating the United States and claiming priority from German patent application DE 10 2019 135 508.7, filed Dec. 20, 2019, and the entire content of both applications is incorporated herein by reference.

### TECHNICAL FIELD

The present disclosure relates to an intraocular lens system, an intraocular lens, and a ciliary body implant. Consequently, the disclosure is in the field of intraocular lenses in particular, more particularly in the field of biomechanically and/or electromechanically accommodatable intraocular lenses.

### BACKGROUND

The related art has disclosed intraocular lenses (IOLs) that exhibit an ability to biomechanically accommodate, that is to say the refractive power of the IOL can be changed by a mechanical force being exerted by means of muscular tissue and can be adapted to the desired accommodation.

IOLs are frequently implanted into the capsular bag of the eye as this has a low complication rate in comparison with other implantation locations, and the required surgical techniques are well engineered, with numerous concepts for such biomechanical IOLs being available. For the accommodation, the concepts known from the related art use the naturally triggering force, specifically the change in diameter of the ciliary body or ciliary muscle, only indirectly. Rather, the decisive force transmission is implemented onto the elastic capsular bag via the zonular fibers.

In different eyes or patients, the elasticity of the capsular bag is very different on an individual basis and may change, for example as a result of wound healing processes (e.g., fibrosis) following cataract surgery and as a result of further cell growth (secondary cataract). The treatment of the secondary cataract may also change the capsular bag and, in particular, the elasticity thereof. It is therefore often difficult to find a generally valid, well-functioning biomechanical arrangement which is equally suited to many individual differences in the population and, additionally, to the temporal changes of the biological material used for the function. What makes the matter more difficult is that the properties of the biological material used for the function typically cannot be measured before a cataract operation, which is why it is not possible to adapt to individual conditions.

Moreover, conventional IOL implants typically must span the original capsular bag in order to reduce/avoid fibrosis, necessitating a large implant volume and making small incision dimensions more difficult.

Furthermore, there exist concepts for biomechanical accommodatable IOLs which are implanted into the sulcus, or into the vicinity of the sulcus, outside of the capsular bag, in direct contact with the ciliary muscle. In this case, the strength of the ciliary muscle is directly converted into a mechanical movement or hydraulic deformation in order to generate an accommodation of the biomechanical implant. These implants are typically in contact with the iris or carry out relative movements in relation to critical tissue, as a result of which for example pigments can be detached from the iris and these can then for example impede the drainage of the eye fluid. What are known as sulcus IOLs exhibit an elevated complication rate as a result of this and other effects.

By way of example, U.S. 2013/0226293 A1 describes an electroactive IOL, which is directly mechanically connectable to the ciliary muscle. Consequently, such an IOL does not facilitate a complete implantation into the capsular bag.

Further, WO 2015/138507 A1 discloses a sensor cell for implantation into the ciliary muscle, the sensor cell having a pressure-sensitive or electromyographic form and comprising a microchip for active, wireless transmission of the signal to an optically effective implant. Since the sensor cell has an active design, it requires a suitable power supply and must be encapsulated in hermetically sealed fashion in relation to its surroundings.

U.S. 2014/0156000A1 describes an electroactive ophthalmic lens having an electromyography sensor, a processor, and an electroactive optical element. The electromyography sensor is designed to detect an electric field in the ciliary muscle, the electric field being proportional to the force exerted by the ciliary muscle, and to generate a sensor signal indicative therefor. The electromyographic signals of the electric fields in the ciliary muscle are typically very weak and moreover can only be separated with difficulty from the electromyographic signals of other, larger muscles (for example, the muscles for moving the eye), making a reliable use of these signals more difficult.

### SUMMARY

It is an object of the present disclosure to provide an intraocular lens system which avoids the disadvantages afflicting the conventional IOLs.

According to the disclosure, this object is achieved by an intraocular lens system, a ciliary body implant, an intraocular lens, and a method for implanting an intraocular lens system, wherein the refractive power of an intraocular lens is controlled on the basis of a ciliary signal received a the sensor element.

In a first aspect, the disclosure relates to an intraocular lens system for implantation into an eye. The intraocular lens system comprises a ciliary body implant having a passive ciliary signal element, the ciliary body implant being designed and being implantable into the eye in such a way that the ciliary signal element provides a ciliary signal on the basis of a movement of the ciliary muscle of the eye. Moreover, the intraocular lens system comprises an intraocular lens comprising a sensor element for receiving the ciliary signal. Here, the ciliary body implant and the intraocular lens are formed separately from one another and the intraocular system is further designed to control a refractive power of the intraocular lens on the basis of the ciliary signal received by the sensor element.

In a further aspect, the disclosure relates to a ciliary body implant for an intraocular lens system for implantation into an eye, the ciliary body implant comprising a passive ciliary signal element and being designed to provide, by means of the ciliary signal element, a ciliary signal on the basis of a movement of the ciliary muscle of the eye.

In a further aspect, the disclosure relates to an intraocular lens for an intraocular lens system for implantation into an eye, the intraocular lens comprising a sensor element for receiving a ciliary signal and being designed to control a refractive power of the eye on the basis of the received ciliary signal.

In a further aspect, the disclosure relates to a method for implanting an intraocular lens system into an eye. The method comprises an implantation of a ciliary body implant having a passive ciliary signal element into the eye, in such a way that the ciliary signal element provides a ciliary signal on the basis of a movement of the ciliary muscle of the eye. Further, the method comprises an implantation of an intraocular lens into the eye, the intraocular lens comprising a sensor element for receiving the ciliary signal. Here, the ciliary body implant and the intraocular lens are formed separately from one another and the intraocular system is further designed to control a refractive power of the intraocular lens on the basis of the ciliary signal received by the sensor element.

Within the meaning of the disclosure described here, an intraocular lens system is a system comprising a biomechanically and/or electroactively accommodatable intraocular lens (IOL) and one or more further elements for detecting the desire to accommodate, such as the ciliary body implant in particular, and for implementing the accommodation of the IOL ciliary body implant. In this case, the intraocular lens system (IOL system) according to the disclosure has a multi-part design, with the plurality of parts of the IOL system being available as separate parts and, in particular, being implantable into the eye separately from one another. Typically, the plurality of a parts of the IOL system, in particular the IOL and the ciliary body implant, require no direct mechanical and/or hydraulic and/or “wired” electrical connection to one another.

In this case, the ciliary body implant is an implant that is implantable into the eye and that at least partly follows the movements of the ciliary muscle. In this case, it is not mandatory for the ciliary body implant to be implanted and/or arranged directly in and/or on the ciliary muscle. Rather, indirect mechanical contact between the ciliary body implant and the ciliary muscle of the eye may also be sufficient for as long as the ciliary body implant at the implanted site at least partly follows the movements of the ciliary muscle. In this case, the ciliary body implant typically fulfills the function of generating a signal from the movements of the ciliary muscle, the signal indicating the desire to accommodate and being able to be used for the accommodation of the IOL or of the eye. In this case, the ciliary body implant may be arranged in direct contact with the ciliary muscle and/or with the ciliary body. By way of example, the ciliary body implant can be arranged in direct mechanical contact with the ciliary body and can be in indirect contact with the ciliary muscle via the ciliary body.

In this case, the ciliary signal element is a passive signal generator, which is integrated into the ciliary body implant and/or connected to the latter, and which is designed, on the basis of a movement of the ciliary muscle of the eye, to provide a signal for the indication of the desire to accommodate. By way of example, this can be implemented by virtue of the ciliary signal element at least partly following the movements of the ciliary muscle and/or by virtue of movements of the ciliary muscle exerting a force on the ciliary signal element and/or exerting an influence on the ciliary signal element in any other way. In this case, the ciliary signal element optionally at least partly following the movements of the ciliary muscle means that a deflection of the ciliary signal element or the change in position thereof in the eye need not necessarily be with the same amplitude and/or in the same direction as the deflection of the ciliary muscle which causes the deflection and/or the change in position of the ciliary muscle. Rather, it may be sufficient here for the ciliary signal element to follow the movements of the ciliary muscle in such a way that the ciliary signal element provides a signal which allows at least qualitative identification of the movement of the ciliary muscle. Typically, the signal provided by the ciliary signal element is proportional, typically directly proportional, to the amplitude of the causal movement of the ciliary muscle.

According to the disclosure, the ciliary signal element has a passive design. This means that the ciliary signal element requires no power supply such as a current supply. Typically, this further means that the ciliary signal element comprises no circuits that actively generate a signal. Rather, the ciliary signal element is designed to generate the signal passively, for instance by virtue of a reaction being caused in the sensor element by means of an electric, more particularly electrostatic, and/or (permanent and/or static) magnetic field emanating from the ciliary signal element. By way of example, the signal at the position of the sensor element can be caused by virtue of the ciliary signal element moving and the electric and/or magnetic field emanating from the ciliary signal element changing at the position of the sensor element as a result. Alternatively or in addition, the signal may for example be passively provided in such a way that electromagnetic radiation, for instance light in the visible and/or invisible spectral range, incident on the ciliary signal element is at least partly reflected and/or scattered and/or refracted and/or diffracted to the sensor element by way of the ciliary signal element. Typically, the ciliary body implant comprises a plurality of passive ciliary signal elements which are arrangeable so as to be spaced apart from one another and in mechanical contact with the ciliary muscle and/or with the sulcus.

In this case, the IOL is an accommodatable IOL, particularly typically a biomechanically and/or electroactively accommodatable IOL. Thus, the eye can accommodate, in particular by way of a change in the refractive power of the IOL in the eye. In this case, the change in the refractive power can typically be implemented by virtue of a mechanical force being exerted on the IOL or at least onto a part of the IOL. In this case, the IOL can typically be designed in such a way that the latter can actively bring about a change in the refractive power of the IOL. The intraocular lens is typically implantable into the capsular bag of the eye.

In this case, the sensor element typically has an active design and typically comprises a suitable power supply, in particular a current supply, such as a battery and/or a rechargeable battery and/or a photovoltaic element, which facilitates the active operation of the sensor element. In this case, the sensor element can receive the ciliary signal and optionally transmit other signals. Typically, the sensor element can be designed to transmit a signal pulse and to detect an echo reflected by the ciliary signal element as a ciliary signal. Typically, the sensor element can be designed to transmit the signal pulse in the form of an optical signal pulse and/or as a radio signal pulse.

In this case, the interaction between the ciliary signal element and the sensor element controlling the refractive power of the IOL means that, typically, a change in the refractive power of the IOL follows a change in the interaction between the ciliary signal element and the sensor element, in particular a change in the magnetic interaction between the ciliary signal element and the sensor element. A movement of the ciliary muscle can thus typically be used to control the refractive power of the IOL and particularly typically be used to provide the force for changing the refractive power of the IOL. Typically, the refractive power of the intraocular lens is controlled on the basis of a change in the ciliary signal at the position of the sensor element in the eye.

The disclosure offers the advantage that the intraocular lens system can be provided with a passive ciliary body implant. This offers the advantage that there is no need to provide a power storage unit, for instance a rechargeable battery and/or a battery, in the ciliary body implant and, accordingly, there also is no need to replace or exchange such a power storage unit. This also offers the advantage that the ciliary body implant typically can be produced from electrically passive materials, thus allowing the production complexity and/or the production costs to be kept low. This also offers the advantage that typically biocompatible materials and/or materials that endure in the environment of the implant are able to be used for the production of the ciliary body implant, allowing the risk of complications to be reduced.

The disclosure also offers the advantage that the IOL system can be implanted into an eye in such a way that there is no need for direct contact between the IOL system and the iris and/or no need for relative movement between the IOL system, in particular the ciliary body implant, and critical tissue in the eye. Complications can thus be avoided since the IOL system for example does not detach pigments from the iris and consequently there is no obstacle for the drainage of the eye fluid through the IOL system. Consequently, the IOL system according to the disclosure allows a reduction in the complication rate in comparison with conventional accommodatable IOLs.

Moreover, the disclosure offers the advantage that the accommodatable IOL can be designed in a compact form and, in particular, an implantation of the IOL into the capsular bag is facilitated and this represents an exemplary embodiment, likewise promoting a low complication rate. This is moreover promoted by the fact that the IOL system according to the disclosure requires no direct mechanical and/or electrical connection between the ciliary body implant and the IOL, and accordingly there is no need to run mechanical connections or electrical conductors from the IOL to the ciliary body implant through the capsular bag. This is advantageous since damage to the capsular bag and possible complications accompanying this can be avoided or reduced. Moreover, this offers the advantage that it is sufficient to merely implant the IOL into the capsular bag and there is no need or cause to implant the ciliary body implant into the capsular bag. In this way, the incision in the capsular bag required for the implantation of the IOL into the capsular bag can be kept small.

Further, the disclosure offers the advantage that no electromyographic signals that are based on electric fields in the ciliary muscle need to be used for the provision of the ciliary signal as these are typically very weak and moreover superposed by other electric fields. This offers the advantage of being able to dispense with complex isolation and/or preparation of an electromyographic signal, which typically requires a floating instrument amplifier with a high input resistance.

Typically, the ciliary body implant is implantable into the eye in such a way that the ciliary signal element is in mechanical contact with the ciliary body and/or with the ciliary muscle and/or with the sulcus. By way of example, the ciliary body implant can be fastened directly on and/or in the ciliary body and/or can be positioned in the sulcus and/or in the vicinity of the sulcus. This offers the advantage that an avoidance of mechanical contact between the ciliary body implant and the iris can be attained particularly reliably. This also offers the advantage that the ciliary signal element can follow the movements of the ciliary muscle and/or ciliary body particularly reliably without, for example, there being a falsification by the zonular fibers and/or the capsular bag. The method for implanting the IOL system is typically implemented in such a way here that the ciliary signal element is in mechanical contact with the ciliary muscle and/or with the ciliary body and/or with the sulcus.

Typically, the ciliary signal element comprises a permanent magnet or is designed as such. Particularly typically, the ciliary signal element is designed to provide the ciliary signal at the position of the sensor element in the eye by means of a magnetic field. This offers the advantage that the ciliary signal can be provided easily and reliably by way of passive means, for example by way of a permanent magnet. Further, this offers the advantage that the surrounding tissue, for instance the zonular fibers and/or the capsular bag, do not bring about relevant attenuation and/or falsification of the magnetic field and, accordingly, the ciliary signal is not substantially attenuated and/or falsified in this way either.

Alternatively or in addition, the ciliary signal element comprises an electrode and/or a piezo element, or is designed as such. Particularly typically, the ciliary signal element is designed to provide the ciliary signal at the position of the sensor element in the eye by means of an electric field. This offers the advantage that the ciliary signal can be reliably passively provided using simple means, for example as an electric field of electrostatic charges on the electrode. Alternatively or in addition, the electric field can be provided by means of a piezo element by way of the ciliary muscle and/or the ciliary body applying a force on the piezo element. When the ciliary signal element changes its position and/or when there is any other change in the electric field on account of a movement of the ciliary muscle and/or ciliary body, this may subsequently cause a change in the electric field at the position of the sensor element.

Alternatively or in addition, the ciliary signal element comprises one or more surface wave structures and is designed to change a characteristic property of the surface wave structure(s) on the basis of mechanical action on the ciliary signal element. By way of example, the one or more surface wave structures may serve to receive an incident electromagnetic wave, for instance a radio signal, and to re-transmit or reflect this in a modified manner. In this case, the manner in which the incident electromagnetic wave is changed is determined by the characteristic property of the surface wave structure, by virtue of the surface wave structure typically at least partly guiding and reflecting the incident electromagnetic wave.

In this case, the change in the characteristic property of the respective surface wave structure is able to be influenced by mechanical action on the ciliary signal element, and so an application of force on the ciliary signal element by the ciliary muscle and/or ciliary body, for instance a compressive and/or tensile and/or shearing force, leads to a change in the surface wave structure which in turn changes the characteristic property of the surface wave structure. In this way, the ciliary signal element can provide a ciliary signal which corresponds to a reflection of an incident electromagnetic wave, the reflection depending on the present application of force on the ciliary signal element by the ciliary muscle and/or ciliary body.

Alternatively or in addition, the ciliary signal element comprises an optical element or is designed as such. Particularly typically, the ciliary signal element is designed to provide the ciliary signal at the position of the sensor element in the eye by means of an optical signal, the optical element typically comprising a mirror and/or a diffractive structure and/or a holographic structure. This offers the advantage that the ciliary signal can be provided passively as an optical signal. To this end, an optical signal, for example, can be transmitted from the sensor element and/or another radiation source in the eye to the ciliary signal element, which then reflects and/or scatters and/or diffracts the optical signal to the sensor element. Alternatively or in addition, the ciliary signal element can typically be embodied in such a way that the ciliary signal is provided by means of reflection and/or scattering and/or diffraction of light incident in the eye. By way of example, a small portion of the incident light can be used for the provision of the ciliary signal without this significantly impairing the transmission of the eye, that is to say the optical components of the eye for a transmission of the light to the retina. By way of example, light in a very tightly bounded spectral range can be diverted to the sensor element to this end. Alternatively or in addition, the ciliary signal element can be designed to provide light in a spectral range that is invisible to the eye, for example light in the infrared spectral range, as a ciliary signal. This offers the advantage that there is no restriction and/or attenuation of light that can be detected by the retina. The provision of the ciliary signal is in this case typically implemented in such a way that an action of the ciliary signal on the ciliary signal element can be recognized on the basis of a property of the ciliary signal provided at the sensor element, for example on the basis of a change in the angle of incidence and/or a signal strength or intensity and/or a wavelength of the ciliary signal.

Typically, the sensor element comprises a solenoid and is particularly typically designed to inductively receive the ciliary signal. A ciliary signal element which comprises a permanent magnet or is designed as such is particularly typically used in this case. This offers the advantage that the magnetic field generated by the permanent magnet can be used to provide the ciliary signal. This in turn offers the advantage that a passive provision of the ciliary signal is made possible in a simple manner. Further, this offers the advantage that the surrounding tissue in the eye does not cause a substantial attenuation of the magnetic field and consequently the ciliary signal can be reliably provided at the position of the sensor element.

Alternatively or in addition, the sensor element typically comprises an electrode and is particularly typically designed to capacitively receive the ciliary signal. By way of example, the ciliary signal element can likewise comprise an electrode or be designed as such. By way of example, the electrode of the ciliary signal element and the electrode of the sensor element can together form a capacitor. In this case, a change in the position of the ciliary signal element on account of a movement of the ciliary muscle and/or ciliary body can typically lead to a change of the capacitance of the capacitor, on the basis of which the causal movement of the ciliary muscle can be reliably detected and a corresponding intention of the eye to accommodate can be reliably recognized.

Alternatively or in addition, the sensor element comprises an electromagnetic resonant circuit or is designed as such. According to some embodiments, one or more components of the ciliary signal may also contribute to the function of the resonant circuit in this case. By way of example, an electrode of the ciliary signal element can serve as a capacitor plate of the capacitor of the resonant circuit. In this case, the sensor element is particularly typically designed to receive the ciliary signal inductively and/or capacitively. A ciliary signal element which comprises a permanent magnet and/or an electrode, or is designed as a permanent magnet or as an electrode, is particularly typically used in this case. As a result, it is possible for example to change the inductive and/or capacitive properties of the resonant circuit by way of a change in position of the ciliary signal element, and the changes can be detected very sensitively and accurately. By way of example, a ciliary signal element designed as an electrode can form the capacitor of the resonant circuit of the sensor element together with a further electrode in the sensor element. A change in the position of the electrode of the ciliary signal element can then lead to a change in the capacitive property of the resonant circuit and can facilitate a reliable and sensitive detection of the causal change in position of the ciliary signal element. This consequently offers the advantage that a change in the ciliary signal, and accordingly a change in the position of the ciliary muscle, can be detected particularly sensitively, and accordingly an intention of the eye to accommodate can be recognized particularly reliably.

Alternatively or in addition, the sensor element can typically comprise a transceiver unit for radio signals or be designed as such. Expressed differently, the sensor element according to exemplary embodiments is designed to transmit and receive radio signals. Particularly typically, such a sensor element is combined with one or more ciliary signal elements which each comprise one or more surface wave structures for the modified reflection of the radio signal transmitted by the sensor element. If the sensor element then transmits a radio signal to the ciliary signal element, the radio signal propagates in the surface wave structure, is modified in accordance with the characteristic properties of the surface wave structure as a result, and is reflected back to the sensor element. On the basis of the reflected radio signal that was modified in accordance with the characteristic properties, the sensor element according to these exemplary embodiments is able to determine whether a force is applied to the ciliary signal element by the ciliary muscle and/or ciliary body and is able to recognize the possible presence of a corresponding desire of the eye to accommodate.

Typically, the ciliary body implant comprises a plurality of passive ciliary signal elements which are elastically interconnected and are arranged in the ciliary body implant in ring-shaped or circular segment-shaped fashion and/or opposite one another relative to the optical axis of the intraocular lens. Particularly typically, the ciliary body implant is designed in ring-shaped or circular segment-shaped fashion and a diameter and/or radius of curvature of the ciliary body implant is changeable by means of the elastic connections between the ciliary signal elements and is typically adaptable to match the ciliary body and/or ciliary muscle. This offers the advantage that the ciliary body implant, in terms of its dimensions, can be adapted to match the inner circumference of the ciliary body and is accordingly arrangeable with precise fit in and/or on the ciliary body and/or on the sulcus.

Typically, the ciliary body implant is implantable into the eye in such a way that there is no direct mechanical contact between the ciliary body implant and the iris of the eye. This offers the advantage of being able to avoid complications, in particular those that arise on account of contact between an IOL and the iris and/or a relative movement of an IOL in relation to sensitive tissue. In particular, this can avoid the risk of pigments detaching from the iris and an impediment to the drainage of the eye fluid caused thereby.

Typically, the intraocular lens comprises an optically transparent lens body and at least one extension, on and/or in which the at least one sensor element is arranged. Typically, the extension comprises a haptic or is designed as such. By way of example, the at least one extension can extend radially outwardly from the lens body. By way of example, the lens extension can be formed lying in the same plane as the lens body. The extension offers the advantage that the sensor element can be arranged in and/or on the IOL without the sensor element covering part of the aperture of the IOL. The sensor element is typically housed in the haptic. The haptic is typically embodied in such a way that the lens body can be well aligned and fixated in the capsular bag. Moreover, the haptic offers the option of arranging one or more sensor elements in and/or on the haptic and accordingly also of fixating and positioning the sensor elements in the eye together with the haptic.

Typically, the refractive power of the intraocular lens is controlled by virtue of the IOL system moving two or more Alvarez plates relative to one another in the intraocular lens on the basis of the ciliary signal. Alternatively or in addition, the refractive power of the intraocular lens can typically be controlled by virtue of the IOL system changing a shape of a membrane in the intraocular lens on the basis of the ciliary signal. This embodiment can be advantageous for fluid-filled lenses in particular, in which the geometric arrangement of the fluid, and hence the lens shape, can be changed by means of the membrane. Alternatively or in addition, the refractive power of the intraocular lens can typically be controlled by virtue of the IOL system changing a spacing of two optical components of an optical doublet in the intraocular lens on the basis of the ciliary signal. Alternatively or in addition, the refractive power of the intraocular lens can typically be controlled by virtue of the IOL system changing the shape of the intraocular lens, which may be advantageous with thin and/or flexible lenses in particular, on the basis of the ciliary signal. However, in addition to these embodiments explicitly mentioned, other mechanisms which facilitate reliable change in the refractive power of the lens with little force outlay are also usable. Alternatively or in addition, the refractive power of the intraocular lens can typically be controlled by virtue of the IOL system changing a refractive index of the IOL on the basis of the ciliary signal.

The features and embodiments specified above and explained below should not only be considered to be disclosed in the respective explicitly mentioned combinations in this case, but are also comprised by the disclosure in other technically advantageous combinations and embodiments.

### BRIEF DESCRIPTION OF THE DRAWINGS

The disclosure will now be described with reference to the drawings wherein:

FIG. 1 shows a schematic representation of an eye with an implanted intraocular lens system according to an exemplary embodiment, both in a longitudinal sectional view and in a transverse sectional view;

FIGS. 2A and 2B show a ciliary body implant according to an exemplary embodiment in various states of accommodation;

FIG. 3 shows the intraocular lens system according to the exemplary embodiment explained in the previous figures, in two different rotational orientations or relative angular positions in relation to the intraocular lens;

FIG. 4 shows an intraocular lens system according to a further exemplary embodiment; and

FIG. 5 shows a ciliary body implant according to an exemplary embodiment.

### DESCRIPTION OF EXEMPLARY EMBODIMENTS

The same or similar elements in the various embodiments are denoted by the same reference signs in the drawings for reasons of simplicity.

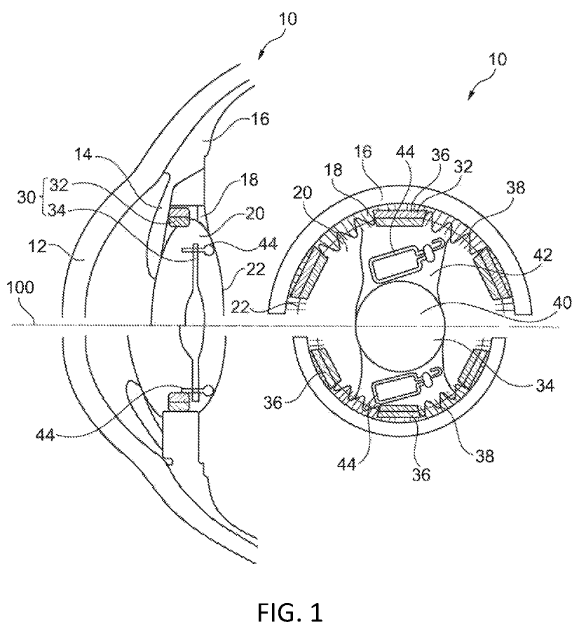

FIG. 1 shows a schematic representation of an eye 10 with an implanted intraocular lens system 30 (IOL system) according to an exemplary embodiment, in a longitudinal sectional view (left) along a sectional plane in which the optical axis 100 of the eye 10 runs, and in a transverse sectional view (right) perpendicular to the optical axis 100.

The longitudinal sectional view of the eye 10 allows identification of the cornea 12 and the iris 14 of the eye 10, and the ciliary muscle or ciliary body 16 located therebehind, the zonula fibers 18 and the empty capsular bag 22, and the space of the removed crystalline lens 20 of the eye 10.

FIG. 1 is also divided in two in the vertical direction, the upper part of the longitudinal sectional view and of the cross-sectional view in each case showing the eye 10 in a first accommodated state and the lower part showing the eye 10 in a second accommodated state. In this case, the first accommodated state can be for example a disaccommodated state of the eye, for example for distance accommodation. By way of example, the second accommodated state can be a significantly accommodated state of the eye 10, for example for near accommodation.

Further, FIG. 1 shows the implanted intraocular lens system 30, which is of multi-part design and which comprises a ciliary body implant 32 and an intraocular lens (IOL) 34, the ciliary body implant 32 and the IOL 34 being formed separately from one another.

According to the exemplary embodiment shown, the ciliary body implant 32 comprises six ciliary signal elements 36, which are elastically interconnected and arranged in such a way that the ciliary body implant 32 is designed as a ring-shaped structure. According to the embodiment shown, the ciliary signal elements 36 are connected by means of mechanical spring elements 38. In this case, the elastic connection of the ciliary signal elements 36 is designed in such a way that a compression and strain of the ciliary body implant 32 is rendered possible in the radial direction such that the ciliary body implant 32 can follow the movements of the ciliary muscle 16 when the eye 10 accommodates or transitions into a non-accommodated state.

According to the embodiment shown, the ciliary signal elements 36 are in the form of permanent magnets and, in this case, arranged in such a way that the magnetic fields of all ciliary signal elements 36 are oriented in the same way in the radial direction. By way of example, all ciliary signal elements 36 can be arranged in such a way that their magnetic south poles point radially inward and their north poles point radially outward. According to other embodiments, the ciliary signal elements 36 can also be arranged in such a way that their magnetic north poles point radially inward and the south poles point radially outward.

In this case, the ciliary body implant 32 is implanted with direct mechanical contact with the ciliary body into the sulcus of the eye 10 or on the sulcus of the eye 10 outside of the capsular bag 22 such that a movement of the ciliary muscle 16 is transferred to the ciliary body implant 32 via the ciliary body and the ciliary body implant accordingly follows the movements of the ciliary muscle 16 by way of a strain or compression. When the following the movements of the ciliary muscle 16, the ciliary body implant 32 can be compressed or strained in such a way by the ciliary muscle 16 or the ciliary body that the diameter of the ciliary body implant 32 increases or reduces, and so the ciliary body implant 32 always rests against the inner side of the ciliary body or against the sulcus.

The IOL 34 is arranged within the capsular bag 22 and comprises a lens body 40 as well as two extensions 42 which contain a haptic. A respective sensor element 44 is arranged in each extension 42. According to other exemplary embodiments, the IOL 34 may also comprise only one or more than two extensions 42, in each of which one or more sensor elements 44 are arranged. The plurality of extensions 42 typically form a haptic.

In this case, the sensor elements 44 each have a solenoid, in which an electric current or any other electric signal can be induced by the magnetic field provided by a respective adjacent ciliary signal element 32, the electric current or other electrical signal then being detectable as ciliary signal by means of the sensor element 44. If the position of the adjacent ciliary signal element 32 changes relative to the sensor element 44, in particular as a result of a movement of the ciliary muscle and/or of the ciliary body, this leads to change in the current induced in the sensor element 44 by the magnetic field of the ciliary signal element 32 and accordingly leads to a change in the ciliary signal.

The ciliary body implant 32 and the IOL 40 are typically arranged in such a way that each sensor element 44 is arranged adjacent to a ciliary signal element 36 in the radial direction in order thus to achieve the greatest possible interaction between the sensor element 44 and the adjacent ciliary signal element 36. In this case, it is advantageous if, like in the embodiment shown, the ciliary body implant 36 comprises a plurality of ciliary signal elements 36, in particular more than two ciliary signal elements 32, since this eases the arrangement of the ciliary body implant 32 and the IOL 34 relative to one another during the implantation, in such a way that a ciliary signal element 36 is arranged adjacent to the respective sensor elements 44 in each case, and hence this simplifies the implantation process.

In this case, the IOL system 30 facilitates a change in the refractive power of the eye 10 on the basis of the ciliary signal. Typically, the IOL system is designed in such a way here that the latter can change the refractive power of the IOL 34 on the basis of the ciliary signal, in such a way that this corresponds to the identified desire to accommodate, which is ascribed to the detected movement of the ciliary muscle 16. Consequently, the implanted IOL system 30 offers the option of changing the refractive power of the IOL 34 by way of the movements of the ciliary muscle 16 and/or the ciliary body, and of accommodating or disaccommodating the eye in this way.

In the upper part of FIG. 1, the eye 10 is depicted in a first accommodated state in each case, which state represents a weak accommodation for distance accommodation. In this case, the ciliary muscle 16 is relaxed and accordingly only a small force is applied to the IOL 34 by the ciliary muscle 16 via the ciliary body, and so the IOL 34 is diametrically relaxed and has a lower refractive power than in a strongly accommodated state. The ciliary body implant 32 is likewise stretched or relaxed in the process and adjusts to the internal diameter of the ciliary body, and so the ciliary body implant 32 also has a larger diameter (relative to the diameter in the unaccommodated state of the eye).

In the lower part of FIG. 1, the eye 10 is depicted in a second accommodated state in each case, which state represents a strong accommodation, for example for near accommodation. In this case, the ciliary muscle 16 is tensioned, as a result of which a radially inwardly directed force indirectly acts on the ciliary body implant 32 and, via the ciliary body implant 32, on the IOL 34. On the basis of the ciliary signal, the IOL system 30 indirectly applies a force at least on the IOL 34, as a result of which the refractive power of the IOL 34 is increased such that the eye 10 accommodates more strongly.

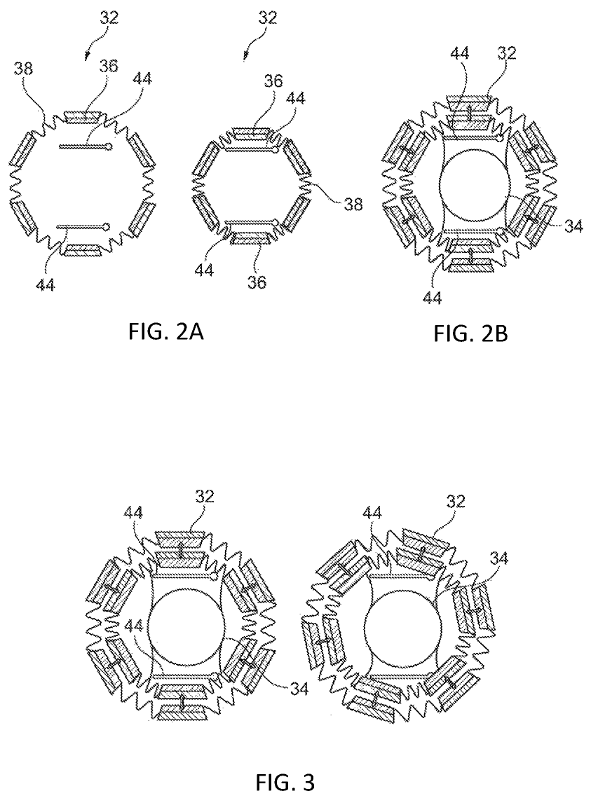

The movement of the ciliary body implant 32 and the resultant change in the relative position of the ciliary signal elements 36 in relation to the sensor elements 44 are shown on the basis of FIGS. 2A and 2B. For a better overview, only the sensor elements 44, and not the other elements of the IOL 34, are depicted. FIG. 2A shows a schematic representation of a ciliary body implant 32 according to the exemplary embodiment shown in FIG. 1. On the right-hand side of FIG. 2A, the ciliary body implant 32 is shown in a radially compressed form, for example in an accommodated state. The left-hand side shows the ciliary body implant 32 in a relaxed or stretched form, for example in a weakly accommodated state of the eye 10. FIG. 2A likewise shows the sensor elements 44 which are located radially within the ciliary body implant 32 and which receive the ciliary signal provided by the respective adjacent ciliary signal element. On account of the compression or relaxation of the ciliary body implant 32, the ciliary signal provided at the position of the sensor element 44 changes, and so the sensor element 44 or the IOL system 40 can on the basis of the respective changes in the ciliary signal determine a movement of the ciliary muscle and accordingly a desire of the eye 10 to accommodate. FIG. 2B elucidates the movement of the ciliary body implant 32 and the change in the relative position of the ciliary signal elements 32 in relation to the sensor elements 44 caused thereby and the resultant change in the ciliary signal on the basis of an overlaid representation of the IOL system in the compressed state (inside) and in the relaxed state (outside).

FIG. 3 shows the IOL system 30 according to the exemplary embodiment explained in the previous figures, in two different rotational orientations or relative angular positions in relation to the IOL 34. In order to obtain the greatest possible interaction between the sensor elements 44 and the respective adjacent ciliary signal elements 36 and, accordingly, in order to maximize an amplitude of the ciliary signal, accurate radially adjacent positioning of one of the ciliary signal elements 36 with respect to the respective sensor elements 44 is advantageous. The use of a large number of ciliary signal elements 34 arranged along the circumferential direction moreover offers the advantage that the requirement of accurate relative positioning of a ciliary signal element 34 with respect to respective sensor element 44 is reduced or dispensed with entirely since the signal field between the individual ciliary signal elements 34 generated from the totality of the ciliary signal elements 34 is not attenuated, or only attenuated to a very small extent, in comparison with positions that directly join a ciliary signal element 36.

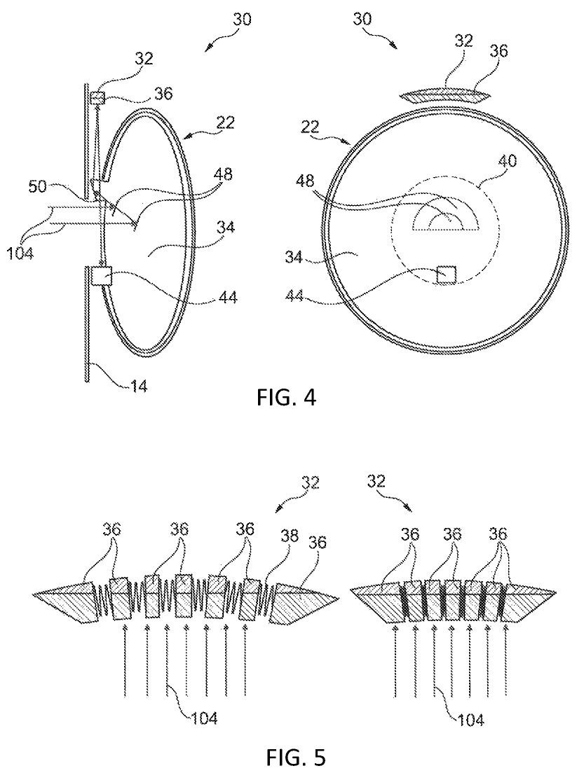

FIG. 4 shows an IOL system 30 according to a further exemplary embodiment. According to this embodiment, the ciliary body implant comprises a ciliary signal element 32 formed as an optical element. The sensor element 44 formed in the IOL 34 behind the iris is designed as an optical sensor. Further, the IOL 34 comprises a plurality of volume hologram elements 48, which are arranged and designed in such a way that these reflect a (small) part of the light incident in the eye 10 toward the ciliary signal element 36. According to the embodiment shown, reflecting the light by the volume hologram elements 48 is implemented by way of a further reflector element 50 formed in or on the IOL 34.

The ciliary body implant 36 may for example be designed as a mirror or comprise the latter. By way of example, the sensor element 44 may comprise a photodetector, for instance a CCD and/or a CMOS detector and/or a photodiode, the photodetector being designed to detect light from the light reflected by the volume hologram elements 48 and the reflective element 50 and typically being designed to convert the light into an electrical signal. By way of example, the volume hologram elements 48 can be designed as a variation of the refractive index of the IOL 34 and be integrated in the lens body 40 of the IOL. In this case, the volume hologram elements 48 are typically designed to reflect light very efficiently in a very tight wavelength range and to transmit light in other wavelength ranges. Typically, the volume hologram elements 48 are designed to reflect light in a spectral range or light with such a wavelength that it is not visible to the eye, that is to say to the retina, in any case, for example light in the infrared spectral range. A reflection of light in the ultraviolet spectral range can typically also be used, provided the lens 20 and the IOL 34 are transparent to the wavelength of the ultraviolet light. Although only one ciliary signal element 36, one sensor element 44, one reflector element 50, and two volume hologram elements 48 are shown, the respective elements may also be present in a different number according to other exemplary embodiments.

Should the ciliary signal element 36 be designed as a mirror or comprise the latter, it may be advantageous to arrange the mirror on the upper side of the ciliary body such that it is directed downward for an upright human. This can reduce or avoid the emergence of deposits on the mirror and an impairment of the functionality accompanying this, in comparison with an arrangement of the mirror on the lower side of the ciliary body with an upward alignment.

The functionality of the IOL system 30 according to this embodiment is based on the fact that a change in distance of the ciliary signal element 36 relative to the sensor element and/or any other change in position of the ciliary signal element 36 caused on account of a movement of the ciliary muscle leads to a change in the luminous flux reaching the sensor element, and this change can be used as a ciliary signal. By way of example, the ciliary signal or its change may consist in a change in the position at which the reflected light strikes the sensor element or the photodetector when the ciliary signal element 36 is moved. By way of example, the sensor element 44 may comprise a position-sensitive photodetector or a two-dimensional detector array to this end. Alternatively or in addition, the intensity or the luminous energy of the light striking the sensor element 44 may change as a result of the change in position of the ciliary signal element 36, and a ciliary signal can be provided on the basis thereof.

FIG. 4 further shows two exemplary beam paths 104 of light incident in the eye. It is evident from these that the light incident into the eye 10 through the pupil in for example collimated fashion is reflected toward the reflector element 50 by the volume hologram elements 48 and is reflected onward to the ciliary signal element 36 from the reflector element. The ciliary signal element 36 in turn reflects light toward the sensor element 44, which then detects the light and determines a ciliary signal therefrom. As a result of the sensor element 44 being arranged behind the iris 14, it is possible to avoid the direct incidence of light on the sensor element 44, that is to say of light that was not reflected by the volume hologram 48 and by the reflector element 50, and a falsification of the ciliary signal caused thereby can accordingly be avoided.

FIG. 5 shows a ciliary body implant 32 according to an exemplary embodiment. The ciliary body implant 32 comprises a total of seven ciliary signal elements 36, each of which is embodied as a mirror element. The ciliary body implant 32 is shown in a stretched state, for example in the case of a relaxed ciliary muscle, on the left-hand side of FIG. 5 and shown in a compressed state, for example in the case of a tensioned ciliary muscle, on the right-hand side. The compression and relaxation of the ciliary body implant 32 can be implemented under compression and relaxation of the mechanical spring elements 38, by means of which the ciliary signal elements 36 are interconnected.

In this case, the ciliary body implant 32 is embodied in such a way that the latter is arrangeable on the inner surface of the ciliary body and/or on the sulcus such that the reflective surfaces are directed inward, that is to say toward the optical axis of the eye.

In this case, the ciliary body implant 32 is embodied in such a way that, in the stretched state, the incident light as represented by the exemplary beam paths 104 partly strikes the ciliary signal elements 36 and is reflected by the latter, while another part of the incident light is incident in the regions between the ciliary signal elements 36 and is accordingly not reflected. As a result of the regions between the ciliary signal elements 36 being greater in the stretched state of the ciliary body implant (to the left in FIG. 5) than in the compressed state (to the right in FIG. 5), a smaller component of the incident light is reflected on the sensor element 44 in the stretched state in comparison with the compressed state. In this way, a change in the intensity of the reflected light can be provided as a ciliary signal. It is understood that the ciliary body implant 32 according to other embodiments may also have a different number of ciliary signal elements 36 and/or in that the ciliary body implant may be designed in a different shape, for example in ring-shaped fashion.

It may be particularly advantageous to combine a ciliary body implant designed in this way and as described in relation to FIG. 5 with an IOL system 30 as described in relation to FIG. 4.

The foregoing description of the exemplary embodiments of the disclosure illustrates and describes the present invention. Additionally, the disclosure shows and describes only the exemplary embodiments but, as mentioned above, it is to be understood that the disclosure is capable of use in various other combinations, modifications, and environments and is capable of changes or modifications within the scope of the concept as expressed herein, commensurate with the above teachings and/or the skill or knowledge of the relevant art.

The term “comprising” (and its grammatical variations) as used herein is used in the inclusive sense of “having” or “including” and not in the exclusive sense of “consisting only of.” The terms “a” and “the” as used herein are understood to encompass the plural as well as the singular.

All publications, patents and patent applications cited in this specification are herein incorporated by reference, and for any and all purposes, as if each individual publication, patent or patent application were specifically and individually indicated to be incorporated by reference. In the case of inconsistencies, the present disclosure will prevail.

### LIST OF REFERENCE SIGNS

10 Eye
12 Cornea
14 Iris
16 Ciliary muscle
18 Zonular fibers
20 Space of the removed crystalline lens of the eye
22 Capsular bag
30 Intraocular lens system (IOL system)
32 Ciliary body implant
34 Intraocular lens (IOL)
36 Ciliary signal element
38 Mechanical spring element
40 Lens body
42 Extension
44 Sensor element
48 Volume hologram element
50 Reflector element
100 Optical axis of the eye
102 Rotational direction for aligning the IOL system
104 Exemplary beam path

1. An intraocular lens system for implantation in an eye, the intraocular lens system comprising:
a ciliary body implant being implantable into the eye and having a passive ciliary signal element, the ciliary body implant being configured such that the passive ciliary signal element provides a ciliary signal based on a movement of a ciliary muscle of the eye; and
an intraocular lens having a sensor element for receiving the ciliary signal,
the ciliary body implant and the intraocular lens being formed separately from one another and the intraocular system being configured to control a refractive power of the intraocular lens based on the ciliary signal received by the sensor element.

2. The intraocular lens system as claimed in claim 1, wherein the ciliary body implant is implantable into the eye such that the ciliary signal element is in mechanical contact with the ciliary body and/or with a sulcus.

3. The intraocular lens system as claimed in claim 1, wherein the intraocular lens is implantable into the capsular bag of the eye.

4. The intraocular lens system as claimed in claim 1, wherein the ciliary signal element comprises a permanent magnet or is formed as the permanent magnet.

5. The intraocular lens system as claimed in claim 1, wherein the ciliary signal element comprises an electrode or is formed as the electrode.

6. The intraocular lens system as claimed in claim 1, wherein the ciliary signal element comprises one or more surface wave structures and is configured to change a characteristic property of the one or more surface wave structures based on a mechanical action on the ciliary signal element.

7. The intraocular lens system as claimed in claim 1, wherein the ciliary signal element comprises an optical element or is formed as the optical element.

8. The intraocular lens system as claimed in claim 1, wherein the sensor element comprises a solenoid and/or an electrode and/or an electromagnetic resonant circuit and/or an optical sensor.

9. The intraocular lens system as claimed in claim 1, wherein the refractive power of the intraocular lens is controlled based on a change in the ciliary signal at a position of the sensor element in the eye.

10. The intraocular lens system as claimed in claim 1, wherein the ciliary body implant comprises a plurality of passive ciliary signal elements which are arrangeable so as to be spaced apart from one another and in mechanical contact with the ciliary body and/or with the sulcus.

11. The intraocular lens system as claimed in claim 10, wherein the plurality of ciliary signal elements is elastically interconnected and is arranged in the ciliary body implant in ring-shaped or circular segment-shaped fashion and/or opposite one another relative to the optical axis of the intraocular lens.

12. The intraocular lens system as claimed in claim 11, wherein the ciliary body implant is configured in ring-shaped or circular segment-shaped fashion and a diameter and/or radius of curvature of the ciliary body implant is changeable via the elastic connections between the ciliary signal elements and is optionally adaptable to match the ciliary body.

13. The intraocular lens system as claimed in claim 1, wherein the ciliary body implant is implantable into the eye such that there is no direct mechanical contact between the ciliary body implant and an iris of the eye.

14. The intraocular lens system as claimed in claim 1, wherein the refractive power of the intraocular lens is controlled in response to the ciliary signal by virtue of the intraocular lens system:
at least partly changing the refractive index of the intraocular lens;
moving two or more Alvarez plates in the intraocular lens relative to one another;
changing a shape of a membrane in the intraocular lens;
changing a distance between two optical components of an optical doublet in the intraocular lens; and/or
changing a shape of the intraocular lens.

15. The intraocular lens system as claimed in claim 4, wherein the ciliary signal element is configured to provide the ciliary signal at the position of the sensor element in the eye via a magnetic field.

16. The intraocular lens system as claimed in claim 5, wherein the ciliary signal element is configured to provide the ciliary signal at the position of the sensor element in the eye via an electric field.

17. The intraocular lens system as claimed in claim 7, wherein the optical element is typically configured to provide the ciliary signal at the position of the sensor element in the eye via an optical signal.

18. The intraocular lens system as claimed in claim 7, wherein the optical element comprises a mirror and/or a diffractive structure and/or a holographic structure.

19. The intraocular lens system as claimed in claim 8, wherein the sensor element is configured to receive the ciliary signal inductively and/or capacitively and/or as an echo of a radio signal.

20. A ciliary body implant for an intraocular lens system for implantation into an eye, the ciliary body implant comprising:
a passive ciliary signal element, the ciliary body implant being configured to provide, via the passive ciliary signal element, a ciliary signal based on a movement of a ciliary muscle of the eye.

21. An intraocular lens for an intraocular lens system for implantation into an eye, the intraocular lens comprising a sensor element for receiving a ciliary signal and being configured to control a refractive power of the eye based on the received ciliary signal.

22. A method for implanting an intraocular lens system into an eye, the method comprising:
implanting a ciliary body implant having a passive ciliary signal element into the eye such that the passive ciliary signal element provides a ciliary signal based on a movement of a ciliary muscle of the eye; and
implanting an intraocular lens into the eye, the intraocular lens having a sensor element for receiving the ciliary signal, the ciliary body implant and the intraocular lens being formed separately from one another, and the intraocular system being configured to control a refractive power of the intraocular lens based on the ciliary signal received by the sensor element.
